(19) United States
(12) Patent Application Publication
(10) Pub. No.: US 2005/0192188 A1
(43) Pub. Date: Sep. 1, 2005

(54) Mild body wash

(76) Inventors: Julie Ann Wagner, Cincinnati, OH (US); Karl Shiqing Wei, Mason, OH (US); Edward Dewey Smith III, Mason, OH (US)

Correspondence Address: THE PROCTER & GAMBLE COMPANY;INTELLECTUAL PROPERTY DIVISION, WINTON HILL TECHNICAL CENTER - BOX 161, 6110 CENTER HILL AVENUE, CINCINNATI, OH, 45224, US

(21) Appl. No.: 11/067,138

(22) Filed: Feb. 25, 2005

Related U.S. Application Data
(60) Provisional application No. 60/548,738, filed on Feb. 27, 2004.

(51) Int. Cl. A61K 7/50

(52) U.S. Cl. 510/130

(57) ABSTRACT

The present invention relates to a mild body wash composition that contains a surfactant component containing a surfactant or a mixture of surfactants; wherein said mild body wash composition has a Structured Domain Volume Ratio of at least about 70%; and where said surfactant component provides a Total Lather Volume of at least about 600 ml.

### CROSS REFERENCE TO RELATED APPLICATION(S)

This application claims the benefit of provisional application No. 60/548,738, filed Feb. 27, 2004.

### FIELD OF THE INVENTION

The present invention relates to a mild body wash composition comprising a surfactant component comprising a surfactant or a mixture of surfactants; wherein said mild body wash composition has a Structured Domain Volume Ratio of at least about 70%; and wherein said surfactant component has a Total Lather Volume of at least about 600 ml.

### BACKGROUND OF THE INVENTION

Personal care composition products like body washes are becoming more popular in the United States and around the world. Desirable body wash compositions must meet a number of criteria. For example, in order to be acceptable to consumers, a body wash composition must exhibit good cleaning properties, must exhibit good lathering characteristics, must be mild to the skin (not cause drying or irritation) and preferably should even provide a conditioning benefit to the skin.

Body wash compositions that attempt to provide skin-conditioning benefits are known. Many of these compositions are aqueous systems comprising an emulsified conditioning oil or other similar materials in combination with a lathering surfactant. Although these products provide both conditioning and cleansing benefits, it is often difficult to formulate a product that deposits sufficient amount of skin conditioning agents on skin during use. In order to combat emulsification of the skin conditioning agents by the cleansing surfactant, large amounts of the skin conditioning agent are added to the compositions. However, this introduces another problem associated with these cleansing and conditioning products. Raising the level of skin conditioning agent in order to achieve increased deposition negatively affects product lather performance and stability.

Accordingly, the need still remains for stable mild body wash composition that provides cleansing with increased lather longevity and improved lathering characteristics, and skin benefits such as silky skin feel, improved soft skin feel, and improved smooth skin feel.

It is therefore an object of the present invention to provide a body wash composition comprising surfactants having a structured domain that can be combined with high levels of skin conditioning materials that are emulsified in the composition, preferably being suspended in such a way so that the skin conditioning materials can be deposited at higher levels while at the same time maintaining superior lather performance versus conventional body washes.

### SUMMARY OF THE INVENTION

The present invention relates to a mild body wash composition comprising a surfactant component comprising a surfactant or a mixture of surfactants; wherein said mild body wash composition has a Structured Domain Volume Ratio of at least about 70%; and wherein said surfactant component has a Total Lather Volume of at least about 600 ml.

The present invention further relates to a mild body wash composition comprising a surfactant component comprising a surfactant or a mixture of surfactants; wherein said surfactant component has at least about 70% of a lamellar phase; and wherein said composition has a Total Lather Volume of at least about 600 ml.

The present invention is also directed to a method of cleansing, moisturizing and delivering skin benefit agents and particles to the skin by applying to the skin a composition as described above.

### DETAILED DESCRIPTION OF THE INVENTION

The mild body wash composition of the present invention comprises a surfactant component comprising a surfactant or a mixture of surfactants; wherein said surfactant mild body wash composition has a Structured Domain Volume Ratio of at least about 70%; and wherein said surfactant component has a Total Lather Volume of at least about 600 ml.

These and other essential limitations of the compositions and methods of the present invention, as well as many of the optional ingredients suitable for use herein, are described in detail hereinafter.

The term “anhydrous” as used herein, unless otherwise specified, refers to those compositions or materials containing less than about 10%, more preferably less than about 5%, even more preferably less than about 3%, even more preferably zero percent, by weight of water.

The term “ambient conditions” as used herein, refers to surrounding conditions at one (1) atmosphere of pressure, 50% relative humidity, and 25° C.

The term “cosmetically efficacious level” as used herein, is a level conferring a benefit during use of the composition.

The term “Consistency value” or “k” as used herein is a measure of viscosity and is used in combination with Shear Index, to define viscosity for materials whose viscosity is a function of shear. The measurements are made at 25° C. and the units are poise (equal to 100 centipoise).

As used herein “domain” means a volume of material, component, composition or phase comprising a molecular mixture which can be concentrated but not further separated by physical forces such as ultracentrifugation. For example, surfactant lamellar, surfactant micellar, surfactant crystal, oil, wax, water-glycerine mixture, hydrated hydrophilic polymer all constitute domains which can be concentrated and observed by ultracentrifugation, but which cannot be further separated into distinct molecular components by the same forces.

The term “hydrophobically modified interference pigment” or “HMIP”, as used herein, means a portion of the interference pigment surface has been coated, including both physical and chemical bonding of molecules, with a hydrophobic material.

The term “interference pigment”, as used herein, means a pigment with pearl gloss prepared by coating the surface of a particle substrate material (generally platelet in shape) with a thin film. The thin film is a transparent or semitransparent material having a high refractive index. The higher refractive index material shows a pearl gloss resulting from mutual interfering action between reflection and incident light from the platelet substrate/coating layer interface and reflection of incident light from the surface of the coating layer.

The term “mild body wash composition” as used herein, refers to compositions intended for topical application to the skin or hair.

The term “opaque” structured surfactant phase as used herein, refers to a surfactant phase with ordered structures (e.g., lamellar structure, vesicule structure, cubic structure, etc.) and it is visually opaque to a naked eye in a 10 mm inner diameter plastic centrifuge tube after the Ultracentrifugation Method described herein.

The term “phases” as used herein, refers to a region of a composition having one average composition, as distinct from another region having a different average composition.

The term “Shear Index” or “n” as used herein, is a measure of viscosity and is used in combination with Consistency value, to define viscosity for materials whose viscosity is a function of shear. The measurements are made at 25° C. and the units are dimensionless.

The phrase “substantially free of” as used herein, means that the composition comprises less than about 3%, preferably less than about 1%, more preferably less than about 0.5%, even more preferably less than about 0.25%, and most preferably less than about 0.1%, by weight of the composition, of the stated ingredient.

The Vaughan Solubility Parameter (VSP) as used herein, is a parameter used to define the solubility of hydrophobic compositions comprising hydrophobic materials. Vaughan Solubility parameters are well known in the various chemical and formulation arts and typically have a range of from about 5 to about 25 (cal/cm³)^{1/2}.

All percentages, parts and ratios as used herein, are by weight of the total composition, unless otherwise specified. All such weights as they pertain to listed ingredients are based on the active level and, therefore do not include solvents or by-products that may be included in commercially available materials, unless otherwise specified.

The mild body wash compositions and methods of the present invention can comprise, consist of, or consist essentially of, the essential elements and limitations of the invention described herein, as well as any additional or optional ingredients, components, or limitations described herein or otherwise useful in personal care compositions intended for topical application to the hair or skin.

### Product Form

The mild body wash composition of the present invention is typically in the form of a liquid. The term “liquid” as used herein means that the composition is generally flowable to some degree. “Liquid”, therefore, can include liquid, semi-liquid, cream, lotion or gel compositions intended for topical application to skin. The compositions typically exhibit a viscosity of equal to or greater than about 1,500 cps to about 1,000,000 cps, as measured by the Viscosity Method as described in copending application Ser. No. 60/542,710 filed on Feb. 6, 2004.

When evaluating a mild body wash compositions comprising a surfactant component and additional benefit material, the body wash can be separated by a separation means, including centrifugation, ultracentrifugation, pipetting, filtering, washing, dilution, or combination thereof, and then the separate components can be evaluated. Preferably, the separation means is chosen so that the resulting separated components being evaluated is not destroyed, but is representative of the component as it exists in the mild body wash composition. All of the product forms contemplated for purposes of defining the compositions and methods of the present invention are rinse-off formulations, by which is meant the product is applied topically to the skin or hair and then subsequently (i.e., within minutes) the skin or hair is rinsed with water, or otherwise wiped off using a substrate or other suitable removal means with deposition of a portion of the composition.

### Surfactant Component

The mild body wash composition of the present invention comprises a surfactant component comprising a surfactant or a mixture of surfactants. The surfactant component comprises surfactants suitable for application to the skin or hair. Suitable surfactants for use herein include any known or otherwise effective cleansing surfactant suitable for application to the skin, and which is otherwise compatible with the other essential ingredients in the mild body wash composition including water. These surfactants include anionic, nonionic, cationic, zwitterionic or amphoteric surfactants, soap or combinations thereof. Preferably the surfactant component comprises a mixture of at least one nonionic surfactant, at least one anionic surfactant and at least one amphoteric surfactant. The general categories of alkyl amines and alkanolamines are less preferred surfactants, because such surfactants tend to be less mild than other suitable surfactants. In a preferred embodiment of the present invention, the mild body wash composition is substantially free of alkyl amines and alkanolamines.

The surfactant component in the present invention exhibits Non-Newtonian shear thinning behavior. Preferably, the mild body wash composition has a viscosity of greater than about 1,500 centipoise (“cps”), more preferably greater than about 5,000 cps, even more preferably greater than about 10,000 cps, and still more preferably greater than about 20,000 cps, as measured by the Viscosity Method described in copending application Ser. No. 60/542710 filed on Feb. 6, 2004.

The surfactant component comprises a structured domain comprising a structured surfactant system. The structured domain enables the incorporation of high levels of benefit components that are emulsified in the composition but suspended. In a preferred embodiment the structured domain is an opaque structured domain. The opaque structured domain is preferably a lamellar phase. The lamellar phase produces a lamellar gel network that is a type of colloidal system. The lamellar phase provides resistance to shear, adequate yield to suspend particles and droplets and at the same time provides long term stability, since it is thermodynamically stable. The lamellar phase yields a higher viscosity without the need for viscosity modifiers.

Preferably, the surfactant component has a Yield Point of greater than about 0.1 Pascal (Pa), more preferably greater than about 0.5 Pascal, even more preferably greater than about 1.0 Pascal, still more preferably greater than about 2.0 Pascal, still even more preferably greater than about 5 Pascal, and even still even more preferably greater than about 10 Pascal as measured by the Yield Point Method described hereafter.

The mild body wash composition comprising the surfactant component has a Structured Domain Volume Ratio of at least about 70%, preferably at least about 75%, more preferably at least about 80%, even more preferably at least about 85% as measured by the Ultracentrifugation Method described hereafter.

The mild body wash composition preferably comprises a surfactant component at concentrations ranging from about 1% to about 95%, preferably 1% to about 80%, more preferably 5% to about 80%, more preferably from about 4% to about 70%, even more preferably from about 5% to about 50%, still more preferably from about 8% to about 30%, and still even more preferably from about 10% to about 25%, by weight of the body wash composition. The preferred pH range of the mild body wash is from about 5 to about 8, more preferably about 6.

The structured domain provides for the incorporation of high levels of benefit components that are not emulsified in the composition but suspended. Even with high levels of benefit components, the mild body wash compositions produce superior lather performance. The surfactant component has a Total Lather Volume of at least about 600 ml, preferably greater than about 800 ml, more preferably greater than about 1000 ml, even more preferably greater than about 1200 ml, still more preferably greater than about 1500 ml, and still even more preferably greater than about 2000 ml, as measured by the Lather Volume Test described hereafter. The surfactant component preferably has a Flash Lather Volume of at least about 300 ml, preferably greater than about 400 ml, even more preferably greater than about 500 ml, as measured by the Lather Volume Test described hereafter

The structured domain has a Total Lather Volume of at least about 450 ml, preferably greater than about 500 ml, more preferably greater than about 600 ml, even more preferably greater than about 800 ml, still more preferably greater than about 1000 ml, and still even more preferably greater than about 1250 ml, as measured by the Lather Volume Test described hereafter. The structured domain preferably has a Flash Lather Volume of at least about 200 ml, preferably greater than about 250 ml, even more preferably greater than about 300 ml, as measured by the Lather Volume Test described hereafter.

Non-Ionic Surfactants

The mild body wash composition preferably comprises at least one nonionic surfactant. Preferably the nonionic surfactant has an HLB from about 1.5 to about 15.0, preferably from about 3.4 to about 15.0, more preferably from about 3.4 to about 9.5, even more preferably from about 3.4 to about 5.0. The mild body wash composition preferably comprises a nonionic surfactant at concentrations ranging from about 0.1% to about 50%, more preferably from about 0.25% to about 30%, even more preferably from about 0.5% to about 25%, still more preferable from about 1.0% to about 20%, and still even more preferably from about 1.5% to about 10%, by weight of the surfactant component.

Non-limiting examples of nonionic surfactants for use in the compositions of the present invention are disclosed in McCutcheon's, Detergents and Emulsifiers, North American edition (1986), published by allured Publishing Corporation; and McCutcheon's, Functional Materials, North American Edition (1992);

Nonionic lathering surfactants useful herein include those selected from the group consisting of alkyl glucosides, alkyl polyglucosides, polyhydroxy fatty acid amides, alkoxylated fatty acid esters, lathering sucrose esters, amine oxides, and mixtures thereof.

Non-limiting examples of preferred nonionic surfactants for use herein are those selected form the group consisting of C₈-C₁₄ glucose amides, C₈-C₁₄ alkyl polyglucosides, sucrose cocoate, sucrose laurate, and mixtures thereof. In a preferred embodiment the nonionic surfactant is selected from the group consisting of glyceryl monohydroxystearate, Steareth-2, propylene glycol stearate, PEG-2 stearate, sorbitan monostearate, glyceryl stearate, laureth-2 and mixtures thereof. In a preferred embodiment the nonionic surfactant is Steareth-2.

Nonionic lathering surfactants also useful herein include, lauramine oxide, cocoamine oxide.

The balance between the hydrophilic and lipophilic moieties in a surfactant molecule is used as a method of classification (hydrophile-lipophile balance, HLB). The HLB values for commonly-used surfactants are readily available in the literature (eg. Handbook of Pharmaceutical Excipients, The Pharmaceutical Press. London, 1994). The HLB system was originally devised by Griffin (J. Soc. Cosmetic Chem., 1, 311, 1949). Griffin defined the HLB value of a surfactant as the mol % of the hydrophilic groups divided by 5, where a completely hydrophilic molecule (with no non-polar groups) had an HLB value of 20.

Anionic Surfactants

The mild body wash composition preferably comprises at least one anionic surfactant. The mild body wash composition preferably comprises an anionic surfactant at concentrations ranging from about 1% to about 50%, more preferably from about 4% to about 30%, even more preferably from about 5% to about 25%, by weight of the surfactant component.

Preferably the anioninc surfactant is selected from the group consisting of alkyl ether sulfates, alkyl sulfonates and mixtures thereof.

Anionic surfactants suitable for use in the mild body wash composition include alkyl and alkyl ether sulfates. These materials have the respective formula ROSO₃M and RO(C₂H₄O)ₓSO₃M, wherein R is alkyl or alkenyl of from about 8 to about 24 carbon atoms, x is 1 to 10, and M is a water-soluble cation such as ammonium, sodium, potassium and triethanolamine. The alkyl ether sulfates are typically made as condensation products of ethylene oxide and monohydric alcohols having from about 8 to about 24 carbon atoms. Preferably, R has from about 10 to about 18 carbon atoms in both the alkyl and alkyl ether sulfates. The alcohols can be derived from fats, e.g., coconut oil or tallow, or can be synthetic. Lauryl alcohol and straight chain alcohols derived from coconut oil are preferred herein. Such alcohols are reacted with about 1 to about 10, preferably from about 3 to about 5, and more preferably with about 3, molar proportions of ethylene oxide and the resulting mixture of molecular species having, for example, an average of 3 moles of ethylene oxide per mole of alcohol, is sulfated and neutralized.

Specific examples of alkyl ether sulfates which may be used in the mild body wash composition are sodium and ammonium salts of coconut alkyl triethylene glycol ether sulfate; tallow alkyl triethylene glycol ether sulfate, and tallow alkyl hexaoxyethylene sulfate. Highly preferred alkyl ether sulfates are those comprising a mixture of individual compounds, said mixture having an average alkyl chain length of from about 10 to about 16 carbon atoms and an average degree of ethoxylation of from about 1 to about 4 moles of ethylene oxide.

Other suitable anionic surfactants include water-soluble salts of the organic, sulfuric acid reaction products of the general formula [R¹—SO₃-M], wherein R¹ is chosen from the group consisting of a straight or branched chain, saturated aliphatic hydrocarbon radical having from about 8 to about 24, preferably about 10 to about 18, carbon atoms; and M is a cation. Suitable examples are the salts of an organic sulfuric acid reaction product of a hydrocarbon of the methane series, including iso-, neo-, ineso-, and n-paraffins, having about 8 to about 24 carbon atoms, preferably about 10 to about 18 carbon atoms and a sulfonating agent, e.g., SO₃, H₂SO₄, oleum, obtained according to known sulfonation methods, including bleaching and hydrolysis. Preferred are alkali metal and ammonium sulfonated C₁₀₋₁₈ n-paraffins.

Other suitable surfactants are described in McCutcheon's, Emulsifiers and Detergents, 1989 Annual, published by M. C. Publishing Co., and in U.S. Pat. No. 3,929,678.

Preferred anionic surfactants for use in the mild body wash composition include ammonium lauryl sulfate, ammonium laureth sulfate, triethylamine lauryl sulfate, triethylamine laureth sulfate, triethanolamine lauryl sulfate, triethanolamine laureth sulfate, monoethanolamine lauryl sulfate, monoethanolamine laureth sulfate, diethanolamine lauryl sulfate, diethanolamine laureth sulfate, lauric monoglyceride sodium sulfate, sodium lauryl sulfate, sodium laureth sulfate, potassium laureth sulfate, sodium lauryl sarcosinate, sodium lauroyl sarcosinate, lauryl sarcosine, cocoyl sarcosine, ammonium cocoyl sulfate, ammonium lauroyl sulfate, sodium cocoyl sulfate, sodium lauroyl sulfate, potassium cocoyl sulfate, potassium lauryl sulfate, monoethanolamine cocoyl sulfate, sodium tridecyl benzene sulfonate, sodium dodecyl benzene sulfonate, and combinations thereof.

Anionic surfactants with branched alkyl chains such as sodium trideceth sulfate, for example, are preferred in some embodiments. Mixtures of anionic surfactants may be used in some embodiments.

Amphoteric Surfactants

The mild body wash composition preferably comprises at least one amphoteric surfactant. The mild body wash composition preferably comprises an amphoteric surfactant at concentrations ranging from about 1% to about 50%, more preferably from about 2% to about 30%, even more preferably from about 3% to about 25%, by weight of the surfactant component.

Amphoteric surfactant suitable for use in the present invention include those that are broadly described as derivatives of aliphatic secondary and tertiary amines in which the aliphatic radical can be straight or branched chain and wherein one of the aliphatic substituents contains from about 8 to about 18 carbon atoms and one contains an anionic water solubilizing group, e.g., carboxy, sulfonate, sulfate, phosphate, or phosphonate. Examples of compounds falling within this definition are sodium 3-dodecyl-aminopropionate, sodium 3-dodecylaminopropane sulfonate, sodium lauryl sarcosinate, N-alkyltaurines such as the one prepared by reacting dodecylamine with sodium isethionate according to the teaching of U.S. Pat. No. 2,658,072, N-higher alkyl aspartic acids such as those produced according to the teaching of U.S. Pat. No. 2,438,091, and the products described in U.S. Pat. No. 2,528,378.

Amphoacetates and diamphoacetates may also be used.

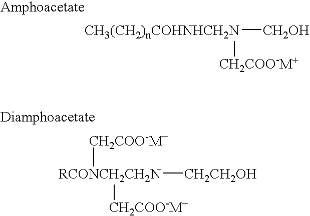

Amphoacetates and diamphoacetates conform to the formulas (above) where R is an aliphatic group of 8 to 18 carbon atoms. M is a cation such as sodium, potassium, ammonium, or substituted ammonium. Sodium lauroamphoacetate, sodium cocoamphoactetate, disodium lauroamphoacetate, and disodium cocodiamphoacetate are preferred in some embodiments.

Additional surfactant from the classes of zwitterionic surfactant, and/or cationic surfactant, may be incorporated in the mild body wash compositions.

Zwitterionic surfactants suitable for use in the mild body wash compositions include those that are broadly described as derivatives of aliphatic quaternary ammonium, phosphonium, and sulfonium compounds, in which the aliphatic radicals can be straight or branched chain, and wherein one of the aliphatic substituents contains from about 8 to about 18 carbon atoms and one contains an anionic group, e.g., carboxy, sulfonate, sulfate, phosphate, or phosphonate. Such suitable zwitterionic surfactants can be represented by the formula: 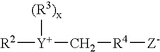
wherein R² contains an alkyl, alkenyl, or hydroxy alkyl radical of from about 8 to about 18 carbon atoms, from 0 to about 10 ethylene oxide moieties and from 0 to about 1 glyceryl moiety; Y is selected from the group consisting of nitrogen, phosphorus, and sulfur atoms; R³ is an alkyl or monohydroxyalkyl group containing about 1 to about 3 carbon atoms; X is 1 when Y is a sulfur atom, and 2 when Y is a nitrogen or phosphorus atom; R⁴ is an alkylene or hydroxyalkylene of from about 1 to about 4 carbon atoms and Z is a radical selected from the group consisting of carboxylate, sulfonate, sulfate, phosphonate, and phosphate groups.

Other zwitterionic surfactants suitable for use in the mild body wash compositions include betaines, including high alkyl betaines such as coco dimethyl carboxymethyl betaine, cocoamidopropyl betaine, cocobetaine, lauryl amidopropyl betaine, oleyl betaine, lauryl dimethyl carboxymethyl betaine, lauryl dimethyl alphacarboxyethyl betaine, cetyl dimethyl carboxymethyl betaine, lauryl bis-(2-hydroxyethyl) carboxymethyl betaine, stearyl bis-(2-hydroxypropyl) carboxymethyl betaine, oleyl dimethyl gamma-carboxypropyl betaine, and lauryl bis-(2-hydroxypropyl)alpha-carboxyethyl betaine. The sulfobetaines may be represented by coco dimethyl sulfopropyl betaine, stearyl dimethyl sulfopropyl betaine, lauryl dimethyl sulfoethyl betaine, lauryl bis-(2-hydroxyethyl) sulfopropyl betaine and the like; amidobetaines and amidosulfobetaines, wherein the RCONH(CH₂)₃ radical is attached to the nitrogen atom of the betaine are also useful in this invention.

Cationic surfactants can also be used in the mild body wash compositions, but are generally less preferred, and preferably represent less than about 5% by weight of the compositions.

Electrolyte

The electrolyte, if used, can be added per se to the composition or it can be formed in situ via the counterions included in one of the raw materials. The electrolyte preferably includes an anion comprising phosphate, chloride, sulfate or citrate and a cation comprising sodium, ammonium, potassium, magnesium or mixtures thereof. Some preferred electrolytes are sodium or ammonium chloride or sodium or ammonium sulfate. A preferred electrolyte is sodium chloride. The electrolyte is preferably added to the surfactant component of the composition.

The electrolyte, when present, should be present in an amount, which facilitates formation of the stable composition (Non-Newtonian shear thinning behavior). Generally, this amount is from about 0.1% by weight to about 15% by weight, preferably from about 1% to about 6% by weight of the composition, but may be varied if required.

Benefit Component

The mild body wash compositions of the present invention can comprise a benefit component. The benefit component is selected from the group consisting of lipids, hydrocarbons, fats, oils, hydrophobic plant extracts, fatty acids, essential oils, silicone materials and mixtures thereof; skin care actives, wherein the skin care actives are selected from the group consisting of vitamins and derivatives thereof; sunscreens; preservatives; anti-acne medicaments; antioxidants; skin soothing and healing; chelators and sequestrants; essential oils, skin sensates, and mixtures thereof.

Hydrophobic Composition

In one preferred embodiment the mild body wash composition comprises a hydrophobic composition comprising a hydrophobic benefit component. The mild body wash compositions comprises from about 20% to about 100%, preferably at least about 35%, most preferably at least about 50% of a hydrophobic benefit component.

The hydrophobic compositions suitable for use in the present invention have a Vaughan Solubility Parameter, as described in copending application Ser. No. 60/542,710 filed on Feb. 6, 2004, of from about 5 to about 15. The hydrophobic compositions are preferably selected among those having defined rheological properties as described in copending application Ser. No. 60/542,710 filed on Feb. 6, 200, including selected Consistency value (k) and Shear Index (n). These preferred Theological properties are especially useful in providing the mild body wash compositions with improved deposition of hydrophobiccomponents on the skin.

Nonlimiting examples of hydrophobic benefit components suitable for use herein can include a variety of hydrocarbons, oils and waxes, silicones, fatty acid derivatives, cholesterol, cholesterol derivatives, diglycerides, triglycerides, vegetable oils, vegetable oil derivatives, acetoglyceride esters, alkyl esters, alkenyl esters, polyglycerin fatty acid esters, lanolin and its derivatives, wax esters, beeswax derivatives, sterols and phospholipids, vitamins and pro-vitamins and combinations thereof.

Non-limiting examples of hydrocarbon oils and waxes suitable for use herein include petrolatum, mineral oil, micro-crystalline waxes, polyalkenes, paraffins, cerasin, ozokerite, polyethylene, perhydrosqualene, and combinations thereof.

Non-limiting examples of silicone oils suitable for use as hydrophobic benefit components herein include dimethicone copolyol, dimethylpolysiloxane, diethylpolysiloxane, mixed C1-C30 alkyl polysiloxanes, phenyl dimethicone, dimethiconol, and combinations thereof. Preferred are non-volatile silicones selected from dimethicone, dimethiconol, mixed C1-C30 alkyl polysiloxane, and combinations thereof. Nonlimiting examples of silicone oils useful herein are described in U.S. Pat. No. 5,011,681 (Ciotti et al.).

Non-limiting examples of diglycerides and triglycerides suitable for use as hydrophobic components herein include castor oil, soy bean oil, derivatized soybean oils such as maleated soy bean oil, safflower oil, cotton seed oil, corn oil, walnut oil, peanut oil, olive oil, cod liver oil, almond oil, avocado oil, palm oil and sesame oil, vegetable oils, sunflower seed oil, and vegetable oil derivatives; coconut oil and derivatized coconut oil, cottonseed oil and derivatized cottonseed oil, jojoba oil, cocoa butter, and combinations thereof.

Non-limiting examples of acetoglyceride esters suitable for use as hydrophobic benefit components herein include acetylated monoglycerides.

Non-limiting examples of alkyl esters suitable for use as hydrophobic benefit components herein include isopropyl esters of fatty acids and long chain esters of long chain (i.e. C₁₀-C₂₄) fatty acids, e.g. cetyl ricinoleate, non-limiting examples of which incloude isopropyl palmitate, isopropyl myristate, cetyl riconoleate and stearyl riconoleate. Other examples are: hexyl laurate, isohexyl laurate, myristyl myristate, isohexyl palmitate, decyl oleate, isodecyl oleate, hexadecyl stearate, decyl stearate, isopropyl isostearate, diisopropyl adipate, diisohexyl adipate, dihexyldecyl adipate, diisopropyl sebacate, acyl isononanoate lauryl lactate, myristyl lactate, cetyl lactate, and combinations thereof.

Non-limiting examples of alkenyl esters suitable for use as hydrophobic benefit components herein include oleyl myristate, oleyl stearate, oleyl oleate, and combinations thereof.

Non-limiting examples of polyglycerin fatty acid esters suitable for use as hydrophobic benefit components herein include decaglyceryl distearate, decaglyceryl diisostearate, decaglyceryl monomyriate, decaglyceryl monolaurate, hexaglyceryl monooleate, and combinations thereof.

Non-limiting examples of lanolin and lanolin derivatives suitable for use as hydrophobic materials herein include lanolin, lanolin oil, lanolin wax, lanolin alcohols, lanolin fatty acids, isopropyl lanolate, acetylated lanolin, acetylated lanolin alcohols, lanolin alcohol linoleate, lanolin alcohol riconoleate, and combinations thereof.

Still other suitable hydrophobic benefit components include milk triglycerides (e.g., hydroxylated milk glyceride) and polyol fatty acid polyesters.

Still other suitable hydrophobic benefit components include wax esters, non-limiting examples of which include beeswax and beeswax derivatives, spermaceti, myristyl myristate, stearyl stearate, and combinations thereof. Also useful are vegetable waxes such as carnauba and candelilla waxes; sterols such as cholesterol, cholesterol fatty acid esters; and phospholipids such as lecithin and derivatives, sphingo lipids, ceramides, glycosphingo lipids, and combinations thereof.

Optional Benefit Components A variety of suitable optional benefit components can be employed in the mild body wash composition. Such benefit components are most typically those materials approved for use in cosmetics and that are described in reference books such as the CTFA Cosmetic Ingredient Handbook, Second Edition, The Cosmetic, Toiletries, and Fragrance Association, Inc. 1988, 1992. These optional benefit components can be used in any aspect of the compositions of the present invention.

Non-limiting optional benefit components include humectants and solutes. A variety of humectants and solutes can be employed and can be present at a level of from about 0.1% to about 50%, preferably from about 0.5% to about 35%, and more preferably from about 2% to about 20%, by weight of the mild body wash composition. A preferred humectant is glycerin.

A preferred water soluble, organic material is selected from the group consisting of a polyol of the structure:

R1-O(CH₂—CR2HO)ₙH

where R1=H, C1-C4 alkyl; R2=H, CH₃ and n=1-200; C2-C10 alkane diols; guanidine; glycolic acid and glycolate salts (e.g. ammonium and quaternary alkyl ammonium); lactic acid and lactate salts (e.g. ammonium and quaternary alkyl ammonium); polyhydroxy alcohols such as sorbitol, glycerol, hexanetriol, propylene glycol, hexylene glycol and the like; polyethylene glycol; sugars and starches; sugar and starch derivatives (e.g. alkoxylated glucose); panthenol (including D-, L-, and the D,L-forms); pyrrolidone carboxylic acid; hyaluronic acid; lactamide monoethanolamine; acetamide monoethanolamine; urea; and mixtures thereof. The most preferred polyols are selected from the group consisting of glycerine, polyoxypropylene(1)glycerol and polyoxypropylene(3)glycerol, sorbitol, butylene glycol, propylene glycol, sucrose, urea and triethanol amine.

Nonionic polyethylene/polypropylene glycol polymers are preferably used as skin conditioning agents. Polymers useful herein that are especially preferred are PEG-2M wherein x equals 2 and n has an average value of about 2,000 (PEG 2-M is also known as Polyox WSR® N-10 from Union Carbide and as PEG-2,000); PEG-5M wherein x equals 2 and n has an average value of about 5,000 (PEG 5-M is also known as Polyox WSR® 35 and Polyox WSR® N-80, both from Union Carbide and as PEG-5,000 and Polyethylene Glycol 200,000); PEG-7M wherein x equals 2 and n has an average value of about 7,000 (PEG 7-M is also known as Polyox WSR® (N-750 from Union Carbide); PEG-9M wherein x equals 2 and n has an average value of about 9,000 (PEG 9-M is also known as Polyox WSR® N-3333 from Union Carbide); PEG-14 M wherein x equals 2 and n has an average value of about 14,000 (PEG 14-M is also known as Polyox WSR-205 and Polyox WSR® N-3000 both from Union Carbide); and PEG-90M wherein x equals 2 and n has an average value of about 90,000. (PEG-90M is also known as Polyox WSR®-301 from Union Carbide.)

Other non limiting examples of these optional benefit components include vitamins and derivatives thereof (e.g., ascorbic acid, vitamin E, tocopheryl acetate, and the like); sunscreens; thickening agents (e.g., polyol alkoxy ester, available as Crothix from Croda); preservatives for maintaining the anti microbial integrity of the cleansing compositions; anti-acne medicaments (resorcinol, salicylic acid, and the like); antioxidants; skin soothing and healing agents such as aloe vera extract, allantoin and the like; chelators and sequestrants; and agents suitable for aesthetic purposes such as fragrances, essential oils, skin sensates, pigments, pearlescent agents (e.g., mica and titanium dioxide), lakes, colorings, and the like (e.g., clove oil, menthol, camphor, eucalyptus oil, and eugenol).

Still other suitable hydrophobic benefit components include ethanol amines of the general structure (HOCH₂CH₂)ₓNH_{y} where x=1-3; y=0-2, and x+y=3.

Particle

The mild body wash composition can comprise a particle. Water insoluble solid particle of various shapes and densities is useful. In a preferred embodiment, the particle tends to have a spherical, an oval, an irregular, or any other shape in which the ratio of the largest dimension to the smallest dimension (defined as the Aspect Ratio) is less than about 10. More preferably, the Aspect Ratio of the particle is less than about 8, still more preferably the Aspect Ratio of the particle is less than about 5.

The particle of the present invention has a particle size (volume average based on the particle size measurement described in copending application Ser. No. 60/542,710 filed on Feb. 6, 2004) of less than about 100 μm, preferably less than about 80 μm, and more preferably the particle size of less than about 60 μm.

The particle of the present invention preferably has a particle size of greater than about 0.1 μm, preferably a particle size of greater than about 0.5 μm, more preferably, a particle size greater than about 1 μm, still more preferably a particle size greater than about 2 μm, even more preferably a particle size greater than about 3 μm, and still even more preferably a particle size greater than about 4 μm.

The particle has a diameter from about 1 μm to about 70 μm, more preferably from about 2 μm to about 65 μm, and even more preferably from about 2 μm to about 60 μm in diameter.

The mild body wash composition of the present invention comprises the particle at a cosmetically efficacious level. Preferably, the particles are present from at least about 0.1% by weight of the composition, more preferably at least about 0.2% by weight of composition, even more preferably at least about 0.5%, still more preferably at least about 1%, and even still more preferably at least 2% by weight of composition. In the mild body wash composition of the present invention, preferably the particles comprises no more than about 50% by weight of composition, more preferably no more than about 30%, still more preferably no more than about 20%, and even more preferably no more than about 10% by weight of composition.

Preferably, the particle will also have physical properties which are not significantly affected by typical processing of the composition. Preferably, a particle having a melting point greater than about 70° C. is used, more preferably having a melting point greater than about 80° C., and even more preferably having a melting point of greater than about 95° C. is used. As used herein, melting point would refer to the temperature at which the particle transitions to a liquid or fluid state or undergoes significant deformation or physical property changes. In addition, many of the particles of present invention are cross-linked or have a cross-linked surface membrane. These particles do not exhibit a distinct melting point. Cross-linked particles are also useful as long as they are stable under the processing and storage conditions used in the making of compositions.

The particles that can be present in the present invention can be natural, synthetic, or semi-synthetic. In addition, hybrid particles can also be present. Synthetic particles can made of either cross-linked or non cross-linked polymers. The particles of the present invention can have surface charges or their surface can be modified with organic or inorganic materials such as surfactants, polymers, and inorganic materials. Particle complexes can be present.

Non limiting examples of natural particles include various precipitated silica particles in hydrophilic and hydrophobic forms available from Degussa-Huls under the trade name Sipemet. Precipitated™, hydrophobic, synthetic amorphous silica, available from Degussa under the trade name Sipernet D11™ is a preferred particle. Snowtex colloidal silica particles available from Nissan Chemical America Corporation.

Nonlimiting examples of synthetic particles include nylon, silicone resins, poly(meth)acrylates, polyethylene, polyester, polypropylene, polystyrene, polyurethane, polyamide, epoxy resins, urea resins, and acrylic powders. Non limiting examples of useful particles are Microease 110S, 114S, 116 (micronized synthetic waxes), Micropoly 210, 250S (micronized polyethylene), Microslip (micronized polytetrafluoroethylene), and Microsilk (combination of polyethylene and polytetrafluoroethylene), all of which are available from Micro Powder, Inc. Additional examples include Luna (smooth silica particles) particles available from Phenomenex, MP-2200 (polymethylmethacrylate), EA-209 (ethylene/acrylate copolymer), SP-501(nylon-12), ES-830 (polymethly methacrylate), BPD-800, BPD-500 (polyurethane) particles available from Kobo Products, Inc. and silicone resins sold under the name Tospearl particles by GE Silicones. Ganzpearl GS-0605 crosslinked polystyrene (available from Presperse) is also useful.

Non limiting examples of hybrid particles include Ganzpearl GSC-30SR (Sericite & crosslinked polystyrene hybrid powder), and SM-1000, SM-200 (mica and silica hybrid powder available from Presperse).

Exfoliant Particle

The exfoliant particle is selected from the group consisting of polyethylene, microcryatalline wax, jojoba esters, amourphors silica, talc, tracalcium orthophosphate, or blends thereof, and the like. The exfoliant particle has a particle size dimension along the major axis of the particle of from about 100 microns to about 600 microns, preferably from about 100 microns to about 300 microns. The exfoliant particle has a hardness of less than about 4 Mohs, preferably less than about 3 Mohs. The hardness as so measured is a criterion of the resistance of a particular material to crushing. It is known as being a fairly good indication of the abrasive character of a particulate ingredient. Examples of materials arranged in increasing order of hardness according to the Moh scale are as follows: h(hardness)-1:talc; h-2: gypsum, rock salt, crystalline salt in general, barytes, chalk, brimstone; h-4: fluorite, soft phosphate, magnesite, limestone; h-5: apatite, hard phosphate, hard limestone, chromite, bauxite; h-6: feldspar, ilmenite, hornblendes; h-7: quartz, granite; h-8: topaz; h-9: corrundum, emery; and h-10: diamond.

Preferably, the exfoliant particle has a color distinct from the cleansing base. The exfoliant particle is preferably present at a level of less than about 10%, preferably less than about 5%, by wt of the composition.

Shiny Particles

The mild body wash composition can comprise a shiny particle. Nonlimiting examples of shiny particles include the following: interference pigment, multi-layered pigment, metallic particle, solid and liquid crystals, or combinations thereof.

An interference pigment is a pigment with pearl gloss prepared by coating the surface of a particle substrate material with a thin film. The particle substrate material is generally platelet in shape. The thin film is a transparent or semitransparent material having a high refractive index. The high refractive index material shows a pearl gloss resulting from mutual interfering action between reflection and incident light from the platelet substrate/coating layer interface and reflection of incident light from the surface of the coating layer. The interference pigments of the mild body wash compositions preferably comprises no more than about 20 weight percent of the composition, more preferably no more than about 10 weight percent, even more preferably no more than about 7 weight percent, and still more preferably no more than about 5 weight percent of the mild body wash composition. The interference pigment of the mild body wash composition preferably comprises at least about 0.1 weight percent of the mild body wash composition, more preferably at least about 0.2 weight percent, even more preferably at least about 0.5 weight percent, and still more preferably at least about 1 weight percent by weight of the composition. When pigment is applied and rinsed as described in the Pigment Deposition Tape Strip Method as described in copending application Ser. No. 60/469,075, filed on May 8, 2003, the deposited pigment on the skin is preferably at least 0.5 μg/cm², more preferably at least 1 μg/cm², and even more preferably at least 5 μg/cm^{2.}

The interference pigments of the present invention are platelet particulates. The platelet particulates preferably have a thickness of no more than about 5 μm, more preferably no more than about 2 μm, still more preferably no more than about 1 μm. The platelet particulates of the preferably have a thickness of at least about 0.02 μm, more preferably at least about 0.05 μm, even more preferably at least about 0.1 μm, and still more preferably at least about 0.2 μm.

The particle size determines the opacity and luster. The particle size is determined by measuring the diameter thickness of the particulate material. The term “diameter” as used herein, means the largest distance across the major axis of the particulate material. Diameter can be determined by any suitable method known in the art, such as particle size analyzer Mastersizer 2000 manufactured by Malvern Instruments. The interference pigment preferably have an average diameter not greater than about 200 μm, more preferably not greater than 100 μm, even more preferably not greater than about 80 μm, still more preferably not greater than than about 60 μm. The interference pigment preferably have a diameter of at least about 0.1 μm, more preferably at least about 1.0 μm, even more preferably at least about 2.0 μm, and still more preferably at least about 5.0 μm.

The interference pigment can comprise a multilayer structure. The centre of the particulates is a flat substrate with a refractive index (RI) normally below 1.8. A wide variety of particle substrates are useful herein. Nonlimiting examples are natural mica, synthetic mica, graphite, talc, kaolin, alumina flake, bismuth oxychloride, silica flake, glass flake, ceramics, titanium dioxide, CaSO₄, CaCO₃, BaSO₄, borosilicate and mixtures thereof, preferably mica, silica and alumina flakes.

A layer of thin film or a multiple layer of thin films are coated on the surface of a substrate described above. The thin films are made of highly refractive materials. The refractive index of these materials is normally above 1.8.

A wide variety of thin films are useful herein. Nonlimiting examples are TiO₂, Fe₂O₃, SnO₂, Cr₂O₃, ZnO, ZnS, ZnO, SnO, ZrO₂, CaF₂, Al₂O₃, BiOCl, and mixture thereof or in the form of separate layers, preferably TiO₂, Fe₂O₃, Cr₂O₃ SnO₂. For the multiple layer structures, the thin films can be consisted of all high refractive index materials or alternation of thin films with high and low RI materials with the high RI film as the top layer.

The interference color is a function of the thickness of thin film, the thickness for a specific color may be different for different materials. For TiO₂, a layer of 40 nm to 60 nm or a whole number multiple thereof gives silver color, 60 nm to 80 nm yellow color, 80 nm to 100 nm red color, 100 nm to 130 nm blue color, 130 nm to 160 nm green color. In addition to the interference color, other transparent absorption pigments can be precipitated on top of or simultaneously with the TiO₂ layer. Common materials are red or black iron oxide, ferric ferrocyanide, chromium oxide or carmine. It was found that the color of the interference pigment in addition to its brightness had a significant influence on human perception of skin tone. In general, preferred colors are silver, gold, red, green and mixtures thereof.

Nonlimiting examples of the interference pigments useful herein include those supplied by Persperse, Inc. under the trade name PRESTIGE®, FLONAC®; supplied by EMD Chemicals, Inc. under the trade name TIMIRON®, COLORONA®, DICHRONA® and XIRONA®; and supplied by Engelhard Co. under the trade name FLAMENCO®, TIMICA®, DUOCHROME®.

In an embodiment of the present invention the interference pigment surface is either hydrophobic or has been hydrophobically modified. The Particle Contact Angle Test as described in copending application Ser. No. 60/469,075, filed on May 8, 2003 is used to determine contact angle of interference pigments. The greater the contact angle, the greater the hydrophobicity of the interference pigment. The interference pigment of the present invention possess a contact angle of at least 60°, more preferably greater than 80°, even more preferably greater than 100°, still more preferably greater than 100°. The hydrophobically modified interference pigment or HMIP allows for the entrapment of the HMIP within the phases and greater deposition of the HMIP. Preferably the ratio of HMIP to a phase is 1:1 to about 1:70, more preferably 1:2 to about 1:50, still more preferably 1:3 to about 1:40 and most preferably 1:7 to about 1:35.

In an embodiment of the present invention the HMIP's are preferably entrapped within the hydrophobic composition. This necessitates that the hydrophobic composition particle size is generally larger than the HMIP. In a preferred embodiment of the invention, the hydrophobic composition particles contain only a small number of HMIPs per hydrophobic composition particles. Preferably this is less than 20, more preferably less than 10, most preferably less than 5. These parameters, the relative size of the benefit droplets to the HMIP and the approximate number of HMIP particles per hydrophobic composition particles, can be determined by using visual inspection with light microscopy.

The HMIP and the hydrophobic composition can be mixed into the composition via a premix or separately. For the case of separate addition, the hydrophobic pigments partition into the hydrophobic composition during the processing of the formulation. The HMIP of the present invention preferably has a hydrophobic coating comprising no more than about 20 weight percent of the total particle weight, more preferably no more than about 15 weight percent, even more preferably no more than about 10 weight percent. The HMIP of the present invention preferably has a hydrophobic coating comprising at least about 0.1 weight percent of the total particle weight, more preferably at least about 0.5 weight percent, even more preferably at least about 1 weight percent. Nonlimiting examples of the hydrophobic surface treatment useful herein include silicones, acrylate silicone copolymers, acrylate polymers, alkyl silane, isopropyl titanium triisostearate, sodium stearate, magnesium myristate, perfluoroalcohol phosphate, perfluoropolymethyl isopropyl ether, lecithin, carnauba wax, polyethylene, chitosan, lauroyl lysine, plant lipid extracts and mixtures thereof, preferably, silicones, silanes and stearates. Surface treatment houses include US Cosmetics, KOBO Products Inc., and Cardre Inc. Yield Point Method

A TA Instruments AR2000 Controlled Stress Rheometer can be used to determine the Yield Point of the surfactant component. For purpose herein, the Yield Point of the surfactant component or the mild body wash composition is the amount of stress required to produce the onset of flow, where a significant increase in strain rate occurs. The determination is performed at 25° C. with a 4 cm diameter parallel plate measuring system and a 1 mm gap. The determination is performed via the programmed application of a shear stress continuous ramp (typically from about 0.1 Pa to about 500 Pa) over a time interval of 5 minutes, collecting 30 data points per decade of stress in an evenly spaced logarithmic stress progression. Stress results in a deformation of the sample, and a shear stress vs. strain curve can be created. The shear stress (Pa) is graphed on the x-axis vs. the strain on the y-axis using logarithmic scales for both axes. The mild body wash composition and surfactant component which are structured exhibit an initial region at low stresses that appears as a straight line when plotted in this way. The Yield Point is the stress point at which the observed strain deviates by greater than 10% from a regression line (i.e. from the predicted strain) extended from the initial straight line region on the log-log plot, determined by linear regression of log-log transformed stress-strain data points between 0.2-2.0 Pa, and continues to deviate by a substantially increasing and accelerating amount with each subsequent point, such that flow occurs. The surfactant component is measured either prior to combining in the composition, or after combining in the composition by separating the compositions by suitable non-destructive physical separation means.

Lather Volume Test

Lather volume of a mild body wash composition, a surfactant component or a structured domain of a mild body wash composition, is measured using a graduated cylinder and a rotating apparatus. A 1,000 ml graduated cylinder is used which is marked in 10 ml increments and has a height of 14.5 inches at the 1,000 ml mark from the inside of its base (for example, Pyrex No. 2982). Distilled water (100 grams at 25° C.) is added to the graduated cylinder. The cylinder is clamped in a rotating device, which clamps the cylinder with an axis of rotation that transects the center of the graduated cylinder. Inject 0.5 cc of a surfactant component or a mild body wash composition into the graduated cylinder onto the side of the cylinder, above the water line, and cap the cylinder. When the structured surfactant domain is evaluated, use only 0.25 cc, keeping everything else the same. The cylinder is rotated for 20 complete revolutions at a rate of about 10 revolutions per 18 seconds, and stopped in a vertical position to complete the first rotation sequence. A timer is set to allow 15 seconds for the lather thus generated to drain. After 15 seconds of such drainage, the first lather volume is measured to the nearest 10 ml mark by recording the lather height in ml up from the base (including any water that has drained to the bottom on top of which the lather is floating).

If the top surface of the lather is uneven, the lowest height at which it is possible to see halfway across the graduated cylinder is the first lather volume (ml). If the lather is so coarse that a single or only a few foam cells (“bubbles”) reach across the entire cylinder, the height at which at least 10 foam cells are required to fill the space is the first lather volume, also in ml up from the base. Foam cells larger than one inch in any dimension, no matter where they occur, are designated as unfilled air instead of lather. Foam that collects on the top of the graduated cylinder but does not drain is also incorporated in the measurement if the foam on the top is in its own continuous layer, by adding the ml of foam collected there using a ruler to measure thickness of the layer, to the ml of foam measured up from the base. The maximum foam height is 1,000 ml (even if the total foam height exceeds the 1,000 ml mark on the graduated cylinder). 30 seconds after the first rotation is completed, a second rotation sequence is commenced which is identical in speed and duration to the first rotation sequence. The second lather volume is recorded in the same manner as the first, after the same 15 seconds of drainage time. A third sequence is completed and the third lather volume is measured in the same manner, with the same pause between each for drainage and taking the measurement.

The lather result after each sequence is added together and the Total Lather Volume determined as the sum of the three measurements, in ml. The Flash Lather Volume is the result after the first rotation sequence only, in ml, i.e., the first lather volume. Compositions according to the present invention perform significantly better in this test than similar compositions in conventional emulsion form.

Ultracentrifugation Method:

The Ultracentrifugation Method is used to determine the percent of a structured domain or an opaque structured domain that is present in a mild body wash composition that comprises a surfactant component. The method involves the separation of the composition through ultracentrifugation into separate but distinguishable layers. The mild body wash composition of the present invention can have multiple distinguishable layers, for example a non-structured surfactant layer, a structured surfactant layer, and a benefit component layer.

First, dispense about 4 grams of body wash product into Beckman Centrifuge Tube (11×60 mm). Next, place the centrifuge tubes in an Ultracentrifuge (Beckman Model L8-M or equivalent) and set ultracentrifuge to the following conditions: 50,000 rpm, 18 hours, and 25° C.

After ultracentrifuging for 18 hours, determine the relative phase volume by measuring the height of each layer using an Electronic Digital Caliper (within 0.01 mm). First, the total height is measured as Hₐ which includes all materials in the ultracentrifuge tube. Second, the height of the benefit component is measured as H_{b}. Third, the height of all structured surfactant layers is measured as H_{c}. The benefit component layer is determined by its low moisture content (less than 10% water as measured by Karl Fischer Titration). It generally presents at the top of the centrifuge tube. The total surfactant layer height (Hₛ) can be calculated by this equation:
Hₛ=Hₐ−H_{b}

The structured surfactant layer components may comprise several layers or a single layer. Upon ultracentrifugation, there is generally an isotropic layer at the bottom or next to the bottom of the ultracentrifuge tube. This clear isotropic layer typically represents the non-structured micellar surfactant layer. The layers above the isotropic layer generally comprise higher surfactant concentration with higher ordered structures (such as liquid crystals). These structured layers are sometimes opaque to naked eyes, or translucent, or clear. There is generally a distinct phase boundary between the structured layer and the non-structured isotropic layer. The physical nature of the structured surfactant layers can be determined through microscopy under polarized light. The structured surfactant layers typically exhibit distinctive texture under polarized light. Another method for characterizing the structured surfactant layer is to use X-ray diffraction technique. The Structured surfactant layer displays multiple lines that are often associated primarily with the long spacings of the liquid crystal structure.

Finally, the structured domain volume ratio is calculated based on the following equation:
Structured Domain Volume Ratio=H_{c}/Hₛ*100%

If there is no benefit component phase present, use the total height as the surfactant layer height, Hₛ=Hₐ.

Method of Use

The mild body wash compositions of the present invention are preferably applied topically to the desired area of the skin or hair in an amount sufficient to provide effective delivery of the surfactant component, hydrophobic benefit material, and particles to the applied surface. The compositions can be applied directly to the skin or indirectly via the use of a cleansing puff, washcloth, sponge or other implement. The compositions are preferably diluted with water prior to, during, or after topical application, and then subsequently the skin or hair rinsed or wiped off, preferably rinsed off of the applied surface using water or a water-insoluble substrate in combination with water.

The present invention is therefore also directed to methods of cleansing the skin through the above-described application of the compositions of the present invention. The methods of the present invention are also directed to a method of providing effective delivery of the desired skin active agent, and the resulting benefits from such effective delivery as described herein, to the applied surface through the above-described application of the compositions of the present invention.

Method of Manufacture

The mild body wash composition of the present invention may be prepared by any known or otherwise effective technique, suitable for making and formulating the desired product form. Specific non-limiting examples of such methods as they are applied to specific embodiments of the present invention are described in the following examples.

It should be understood that every maximum numerical limitation given throughout this specification includes every lower numerical limitation, as if such lower numerical limitations were expressly written herein. Every minimum numerical limitation given throughout this specification includes every higher numerical limitation, as if such higher numerical limitations were expressly written herein. Every numerical range given throughout this specification includes every narrower numerical range that falls within such broader numerical range, as if such narrower numerical ranges were all expressly written herein.

All parts, ratios, and percentages herein, in the Specification, Examples, and Claims, are by weight and all numerical limits are used with the normal degree of accuracy afforded by the art, unless otherwise specified.

### EXAMPLES

The following examples further describe and demonstrate embodiments within the scope of the present invention. The examples are given solely for the purpose of illustration and are not to be construed as limitations of the present invention, as many variations thereof are possible without departing from the spirit and scope of the invention.

### Examples 1-6

The following examples described are non-limiting examples of mild body compositions.

| | Ex. 1 | Ex. 2 | Ex. 3 | Ex. 4 | Ex. 5 | Ex. 6 |
|---|---|---|---|---|---|---|
| | Chemical | Chemical | Chemical | Chemical | Chemical | Chemical |
| CTFA Name | % w/w | % w/w | % w/w | % w/w | % w/w | % w/w |
| Water Distilled | 57.88 | 57.88 | 57.88 | 57.88 | 57.88 | 57.88 |
| Guar | 0.70 | 0.70 | 0.70 | 0.70 | 0.70 | 0.70 |
| hydroxypropyl- | | | | | | |
| trimonium chloride | | | | | | |
| Citric Acid | 1.00 | 1.00 | 1.00 | 1.00 | 1.00 | 1.00 |
| Anhydrous USP | | | | | | |
| Glycerin | 0.30 | 0.30 | 0.30 | 0.30 | 0.30 | 0.30 |
| PEG 90M | 0.10 | 0.10 | 0.10 | 0.10 | 0.10 | 0.10 |
| Citric Acid | 0.09 | 0.09 | 0.09 | 0.09 | 0.09 | 0.09 |
| Anhydrous USP | | | | | | |
| Hampene NA2 | 0.06 | 0.06 | 0.06 | 0.06 | 0.06 | 0.06 |
| (Dissolvine NA- | | | | | | |
| 2X) | | | | | | |
| Sodium Trideceth | 13.00 | 13.00 | 13.00 | 13.00 | 13.00 | 13.00 |
| Sulfate | | | | | | |
| Sodium | 9.20 | 9.20 | 9.20 | 9.20 | 9.20 | 9.20 |
| Lauroamphoacetate | | | | | | |
| Steareth-2 | 1.80 | 0.00 | 0.00 | 0.00 | 0.00 | 0.00 |
| Sorbitan | 0.00 | 1.80 | 0.00 | 0.00 | 0.00 | 0.00 |
| Monostearate | | | | | | |
| Glyceryl | 0.00 | 0.00 | 0.00 | 1.80 | 0.00 | 0.00 |
| Monohydroxy- | | | | | | |
| stearate | | | | | | |
| Glyceryl | 0.00 | 0.00 | 0.00 | 0.00 | 1.80 | 0.00 |
| Monostearate | | | | | | |
| Laureth-2 | 0.00 | 0.00 | 0.00 | 0.00 | 0.00 | 1.80 |
| Propylene Glycol | 0.00 | 0.00 | 1.80 | 0.00 | 0.00 | 0.00 |
| Stearate | | | | | | |
| Petrolatum | 10.00 | 10.00 | 10.00 | 10.00 | 10.00 | 10.00 |
| Sodium Chloride | 3.50 | 3.50 | 3.50 | 3.50 | 3.50 | 3.50 |
| Perfume | 2.00 | 2.00 | 2.00 | 2.00 | 2.00 | 2.00 |
| DMDM Hydantoin | 0.37 | 0.37 | 0.37 | 0.37 | 0.37 | 0.37 |
| | 100.000 | 100.000 | 100.000 | 100.000 | 100.000 | 100.000 |
| Yield Point | 4.6 | 2.4 | 2 | 2 | 1.8 | 3.5 |
| Structured Domain | 79% | 74% | 79% | 81% | 78% | 78% |
| Volume Ratio | | | | | | |
| Total Lather | 1610 | 1870 | 2070 | 1630 | 1790 | 2090 |
| Volume | | | | | | |

The compositions described above can be prepared by conventional formulation and mixing techniques. Combine the following ingredients water distilled, guar hydroxypropyltrimonium chloride, citric acid, anhydrous USP, and glycerin. Heat the mixture to 65-70° C. while agitating the mixture. Keep agitation until a homogenous solution forms. When homogenous, maintain 65-70° C. and add the following ingredients: PEG 90M. Hampene NA2 (Dissolvine NA-2X), sodium trideceth sulfate, sodium lauroamphoacetate, steareth-2. Next, add petrolatum and mix until homogenous. When homogenous, add sodium chloride and mix until homogenous. Adjust the pH to 5.8-6.2 using citric acid. Finally, cool the mixture to 48° C. and add the following ingredients: perfume and DMDM Hydantoin.

The following comparative examples versus the mild-body wash composition described in the present application demonstrate the superior performance the composition of the present invention delivers.

### Comparative Example 1

A body wash is procured having the following ingredients: water, sunflower seed oil, sodium laureth sulfate, sodium lauroamphoacetate, glycerin, petrolatum, lauric acid, cocamide MEA, fragrance, guar hydroxypropyltrimoniumchloride, lanolin alcohol, citric acid, DMDM hydantoin, tetrasodium EDTA, etidronic acid, titanium dioxide, PEG-30 dipolyhydroxystearate. The body wash is marketed under the trade name Dove™ All Day Moisturizing Body Wash by Lever Bros. Co., Greenwich Conn., USA. The body wash contains a Structured Domain Volume Ratio of at least about 42% and has a Total Lather Volume of 1410 ml, and a Flash Lather Volume of 310 ml, and a Yield Stress of 7 Pa.

### Comparative Example 2

A body wash is procured having the following ingredients: water, petrolatum, ammonium laureth sulfate, sodium lauroamphoacetate, ammonium lauryl sulfate, lauric acid, fragrance, trihydroxystearin, citric acid, guar hydroxypropyl trimonium chloride, sodium benzoate, DMDM hydantoin, disodium EDTA, PEG-14M. The body wash is marketed under the trade name Oil of Olay® Daily Renewal Moisturizing Body Wash by Procter & Gamble, Inc., Cincinnati, Ohio, USA. The body wash contains a Structured Domain Volume Ratio of at least about 64% and has a Total Lather Volume of 1630 ml, a Flash Lather Volume of 410 ml, and a Yield Stress of 2.8 Pa.

All documents cited in the Detailed Description of the Invention are, are, in relevant part, incorporated herein by reference; the citation of any document is not to be construed as an admission that it is prior art with respect to the present invention.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

1. A mild body wash composition comprising a surfactant component comprising a surfactant or a mixture of surfactants; wherein said mild body wash composition has a Structured Domain Volume Ratio of at least about 70%; and wherein said surfactant component provides a Total Lather Volume of at least about 600 ml.

2. The mild body wash composition of claim 1, wherein said mild body wash composition has a Structured Domain Volume Ratio of at least about 75%.

3. The mild body wash composition of claim 2, wherein said mild body wash composition has a Structured Domain Volume Ratio of at least about 80%.

4. The mild body wash composition of claim 3, wherein said mild body wash composition has a Structured Domain Volume Ratio of at least about 85%.

5. The mild body wash composition of claim 1, wherein said surfactant component provides a Total Lather Volume of at least about 800 ml.

6. The mild body wash composition of claim 1, wherein said surfactant component provides a Yield Point of greater than about 0.5 Pascal.

7. The mild body wash composition of claim 1, comprising from about 1% to about 95%, by weight of the composition, of said surfactant component.

8. The mild body wash composition of claim 1, wherein said surfactant is selected from the group consisting of anionic surfactant, nonionic surfactant, zwitterionic surfactant, cationic surfactant, amphoteric surfactant, soap, and mixtures thereof.

9. The mild body wash composition of claim 8, wherein said anionic surfactant is selected from the group consisting of alkyl ether sulfates, alkyl sulfonates and mixtures thereof.

10. The mild body wash composition of claim 8, wherein said amphoteric surfactant is selected from the group consisting of sodium lauroamphoacetate, sodium cocoamphoactetate, disodium lauroamphoacetate, and disodium cocodiamphoacetate, and mixtures thereof.

11. The mild body wash composition of claim 8, wherein said nonionic surfactant is selected from the group consisting of glyceryl monohydroxystearate, steareth-2, propylene glycol stearate, sorbitan monostearate, glyceryl stearate, laureth-2, and mixtures thereof.

12. The mild body wash composition of claim 8, comprising from about 0.1% to about 50%, by weight of said surfactant component, of said nonionic surfactant.

13. The mild body wash composition of claim 8, wherein said nonionic surfactant has an HLB of from about 1.5 to about 15.0.

14. The mild body wash composition of claim 1, wherein said composition comprises a structured domain wherein said structured domain is an opaque structured domain.

15. The mild body wash composition of claim 14, wherein said opaque structured domain is a lamellar phase.

16. The mild body wash composition of claim 1, further comprising an electrolyte.

17. The mild body wash composition of claim 1, wherein said composition is substantially free of an alkyl amines and an alkanolamides.

18. The mild body wash composition of claim 1, wherein said composition additionally comprises a benefit component selected from the group consisting of lipids, hydrocarbons, fats, oils, hydrophobic plant extracts, fatty acids, essential oils, silicone materials, vitamins and derivatives thereof; sunscreens; preservatives; anti-acne medicaments; antioxidants; chelators and sequestrants; essential oils, skin sensates, and mixtures thereof.

19. The mild body wash of claim 1, further comprising a particle; wherein said particle is selected from the group consisting of natural, synthetic, semi-synthetic, hybrid and combinations thereof.

20. A mild body wash composition comprising a surfactant component comprising a surfactant or a mixture of surfactants; wherein said composition comprises an opaque structured domain; wherein said opaque structured domain is lamellar phase; wherein said mild body wash composition has a Structured Domain Volume Ratio of at least about 70%; and wherein said surfactant component provides a Total Lather Volume of at least about 600 ml.

21. The mild body wash composition of claim 20, wherein said surfactant is selected from the group consisting of an anionic surfactant, an nonionic surfactant, an zwitterionic surfactant, an cationic surfactant, an amphoteric surfactant, soap, and mixtures thereof.

22. The mild body wash composition of claim 21, wherein said nonionic surfactant has an HLB of from about 3.4 to about 5.0.

23. The mild body wash of claim 20, further comprising a particle; wherein said particle is selected from the group consisting of natural, synthetic, semi-synthetic, hybrid, and combinations thereof.

24. The mild body wash composition of claim 20, wherein said composition additionally comprises a benefit component selected from the group consisting of lipids, hydrocarbons, fats, oils, hydrophobic plant extracts, fatty acids, essential oils, silicone materials; vitamins and derivatives thereof; sunscreens; preservatives; anti-acne medicaments; antioxidants; chelators and sequestrants; essential oils, skin sensates, and mixtures thereof.

25. The mild body wash composition of claim 20, further comprising an electrolyte.

26. The mild body wash composition of claim 20, wherein said composition is substantially free of an alkyl amines and an alkanolamides.

27. A method of delivering skin benefits to skin or hair, said method comprising the steps of:
a) dispensing an effective amount of a mild body wash composition according to claim 1 onto an implement selected from the group consisting of a cleansing puff, washcloth, sponge, and human hand;
b) topically applying said composition to said skin or hair using said implement; and
c) removing said composition from said skin or hair by rinsing said skin or hair with water.
